(19) United States
(12) Patent Application Publication
(10) Pub. No.: US 2026/0098849 A1
(43) Pub. Date: Apr. 9, 2026

(54) SYSTEMS AND METHODS FOR VARNISH DETECTION

(71) Applicant: Donaldson Company, Inc., Bloomington, MN (US)

(72) Inventors: Giancarlo M. Izzi, Minneapolis, MN (US); Michael J. Cronin, Apple Valley, MN (US); Paul J. Michael, Milwaukee, WI (US); Chad M. Goltzman, Bloomington, MN (US); Justin Willmert, Minneapolis, MN (US); James N. Doyle, St. Louis Park, MN (US); David W. Mulder, Bloomington, MN (US); Pawan Panwar, Milwaukee, WI (US)

(21) Appl. No.: 19/349,306

(22) Filed: Oct. 3, 2025

Related U.S. Application Data
(60) Provisional application No. 63/703,728, filed on Oct. 4, 2024.

(51) Int. Cl. G01N 33/28; G01N 9/00; G01N 9/26; G01N 11/04; G01N 27/10; G01N 27/20

(52) U.S. Cl. CPC G01N 33/2805; G01N 9/002; G01N 9/26; G01N 11/04; G01N 27/10; G01N 27/205; G01N 2009/006

(57) ABSTRACT

Embodiments herein relate to systems and methods for varnish detection and/or monitoring in hydrocarbon fluids. In various embodiments, a system for monitoring varnish compounds in fluids is included herein including a control circuit in communication with a fluid property sensor. The system can measure one or more fluid properties (such as dielectric constant, density, resistivity, viscosity and the like) with the fluid property sensor, monitor the same over a time period, and identify an occurrence of a rate of change in the measured property indicating a presence of the varnish compounds. In various embodiments, a system herein can be configured to measure a fluid property of a fluid with a fluid property sensor, monitor the fluid property value over a time period, and predict a time of the onset of varnish compound formation based on the trend of the fluid property value over time. Other embodiments are also included herein.

This application claims the benefit of U.S. Provisional Application No. 63/703,728, filed Oct. 4, 2024, the content of which is herein incorporated by reference in its entirety.

### FIELD

Embodiments herein relate to systems and methods for varnish detection and/or monitoring in hydrocarbon fluids of equipment and systems.

### BACKGROUND

In the context of engine, hydraulic, and other industrial fluid systems, varnish refers to compounds that are oil breakdown products (generated during gradual oxidative and/or thermal breakdown), both soluble and insoluble. Varnish compounds are primarily polar and generally fall out of solution and become deposited on surfaces appearing as amber or brown deposits. As such varnish represents a contaminant in lubricants causing harmful effects on the systems they are deposited in. Over time, varnish accumulation can reduce system performance, increase maintenance costs, and even cause systems to fail, often at great repair and downtime cost.

Currently there is no method to measure or even detect the presence of varnish compounds without taking an oil sample and performing analytical testing in a laboratory setting (e.g., off-line testing).

### SUMMARY

Embodiments herein relate to systems and methods for varnish detection and/or monitoring in hydrocarbon fluids of engine, hydraulic, power generation, transportation, and other industrial fluid systems. In a first aspect, a method of monitoring varnish compounds in fluids is included. The method can include measuring a dielectric constant of a fluid with a fluid property sensor, monitoring the dielectric constant value over a time period, and identifying an occurrence of a rate of change in the dielectric constant value indicating a presence of the varnish compounds.

In a second aspect, in addition to one or more of the preceding or following aspects, or in the alternative to some aspects, the method can further include identifying an occurrence of the rate of change in the dielectric constant value indicating an onset of varnish compound formation.

In a third aspect, in addition to one or more of the preceding or following aspects, or in the alternative to some aspects, the method can further include fitting or modeling the measured dielectric constant value.

In a fourth aspect, in addition to one or more of the preceding or following aspects, or in the alternative to some aspects, wherein fitting or modeling the measured dielectric constant value can be performed using at least one of a polynomial, hyperbolic, exponential, logistic, sigmoid, or an inverse Carreau-Yasuda equation.

In a fifth aspect, in addition to one or more of the preceding or following aspects, or in the alternative to some aspects, the method can further include identifying a period of a linear rate of change in the fluid property followed by a period of a non-linear rate of change in the fluid property.

In a sixth aspect, in addition to one or more of the preceding or following aspects, or in the alternative to some aspects, the method can further include identifying a second period of a linear rate of change in the fluid property following the period of non-linear rate of change.

In a seventh aspect, in addition to one or more of the preceding or following aspects, or in the alternative to some aspects, the fluid property can be dielectric constant.

In an eighth aspect, in addition to one or more of the preceding or following aspects, or in the alternative to some aspects, the method can further include measuring temperature of the fluid with a temperature sensor.

In a ninth aspect, in addition to one or more of the preceding or following aspects, or in the alternative to some aspects, the method can further include normalizing the measured dielectric constant value based on the measured fluid temperature.

In a tenth aspect, in addition to one or more of the preceding or following aspects, or in the alternative to some aspects, the method can further include placing the fluid property sensor after a high heat transfer location in a fluid circuit or at a location with a high differential temperature in a fluid circuit.

In an eleventh aspect, in addition to one or more of the preceding or following aspects, or in the alternative to some aspects, the high heat transfer location in a fluid circuit can be a heat exchanger or cooler in a fluid circuit.

In a twelfth aspect, in addition to one or more of the preceding or following aspects, or in the alternative to some aspects, the fluid property sensor can be a tuning fork-type fluid property sensor.

In a thirteenth aspect, in addition to one or more of the preceding or following aspects, or in the alternative to some aspects, the fluid can be a hydrocarbon fluid.

In a fourteenth aspect, in addition to one or more of the preceding or following aspects, or in the alternative to some aspects, the fluid can be a lubrication fluid.

In a fifteenth aspect, in addition to one or more of the preceding or following aspects, or in the alternative to some aspects, the fluid can be a hydraulic fluid.

In a sixteenth aspect, in addition to one or more of the preceding or following aspects, or in the alternative to some aspects, the fluid can be a gas turbine oil.

In a seventeenth aspect, in addition to one or more of the preceding or following aspects, or in the alternative to some aspects, the method can further include issuing an alert or communication when varnish compounds are detected.

In an eighteenth aspect, in addition to one or more of the preceding or following aspects, or in the alternative to some aspects, the method can further include initiating varnish compound removal and/or mitigation when varnish compounds are detected.

In a nineteenth aspect, in addition to one or more of the preceding or following aspects, or in the alternative to some aspects, the method can further include detecting varnish compound removal and/or remediation after a removal or mitigation operation.

In a twentieth aspect, in addition to one or more of the preceding or following aspects, or in the alternative to some aspects, methos herein can further include measuring a density of the fluid as an additional property with the fluid property sensor, monitoring the density over a time period, and identifying an occurrence of a rate of change in the density value indicating a presence of varnish compounds.

In a twenty-first aspect, in addition to one or more of the preceding or following aspects, or in the alternative to some aspects, methods herein can further include measuring a resistivity of the fluid as an additional property with the fluid property sensor, monitoring the resistivity over a time period, and identifying an occurrence of a rate of change in the resistivity value indicating a presence of varnish compounds.

In a twenty-second aspect, in addition to one or more of the preceding or following aspects, or in the alternative to some aspects, methods herein can further include measuring a viscosity of the fluid as an additional property with the fluid property sensor, monitoring the viscosity over a time period, and identifying an occurrence of a rate of change in the viscosity value indicating a presence of varnish compounds.

In a twenty-third aspect, in addition to one or more of the preceding or following aspects, or in the alternative to some aspects, methods herein can further include measuring a non-fluid property, monitoring the non-fluid property over a time period, and identifying an occurrence of a change in the non-fluid property value indicating a presence of varnish compounds.

In a twenty-fourth aspect, in addition to one or more of the preceding or following aspects, or in the alternative to some aspects, the non-fluid property can include a differential pressure value of a system filter.

In a twenty-fifth aspect, in addition to one or more of the preceding or following aspects, or in the alternative to some aspects, the varnish compounds include hard varnish compounds.

In a twenty-sixth aspect, in addition to one or more of the preceding or following aspects, or in the alternative to some aspects, the varnish compounds include soft varnish compounds.

In a twenty-seventh aspect, a system for monitoring varnish compounds in fluids can be included. The system can include a control circuit and a fluid property sensor. The system for monitoring varnish compounds in fluids can be configured to measure a dielectric constant of a fluid with the fluid property sensor, monitor the dielectric constant over a time period, and identify an occurrence of a rate of change in the dielectric constant value indicating a presence of the varnish compounds.

In a twenty-eighth aspect, in addition to one or more of the preceding or following aspects, or in the alternative to some aspects, the system for monitoring varnish compounds in fluids can be configured to identify an occurrence of the rate of change in the dielectric constant value indicating an onset of varnish compound formation.

In a twenty-ninth aspect, in addition to one or more of the preceding or following aspects, or in the alternative to some aspects, the system for monitoring varnish compounds in fluids can be configured to fit or model the measured dielectric constant value.

In a thirtieth aspect, in addition to one or more of the preceding or following aspects, or in the alternative to some aspects, fitting or modeling the measured dielectric constant value can be performed using at least one of a polynomial, hyperbolic, exponential, logistic, sigmoid, or an inverse Carreau-Yasuda equation.

In a thirty-first aspect, in addition to one or more of the preceding or following aspects, or in the alternative to some aspects, the system for monitoring varnish compounds in fluids can be configured to identify a period of a linear rate of change in the fluid property followed by a period of a non-linear rate of change in the fluid property.

In a thirty-second aspect, in addition to one or more of the preceding or following aspects, or in the alternative to some aspects, the system for monitoring varnish compounds in fluids can be configured to identify a second period of a linear rate of change in the fluid property following the period of non-linear rate of change.

In a thirty-third aspect, in addition to one or more of the preceding or following aspects, or in the alternative to some aspects, can further include a temperature sensor, wherein the system for monitoring varnish compounds in fluids can be configured to measure a temperature of the fluid using the temperature sensor.

In a thirty-fourth aspect, in addition to one or more of the preceding or following aspects, or in the alternative to some aspects, the system for monitoring varnish compounds in fluids can be configured to normalize the measured dielectric constant value based on the measured fluid temperature.

In a thirty-fifth aspect, in addition to one or more of the preceding or following aspects, or in the alternative to some aspects, the fluid property sensor can be positioned after a high heat transfer location in a fluid circuit or at a location with a high differential temperature in a fluid circuit.

In a thirty-sixth aspect, in addition to one or more of the preceding or following aspects, or in the alternative to some aspects, the high heat transfer location can be a heat exchanger or cooler in a fluid circuit.

In a thirty-seventh aspect, in addition to one or more of the preceding or following aspects, or in the alternative to some aspects, the fluid property sensor can be a tuning fork-type fluid property sensor.

In a thirty-eighth aspect, in addition to one or more of the preceding or following aspects, or in the alternative to some aspects, the fluid can be a hydrocarbon fluid.

In a thirty-ninth aspect, in addition to one or more of the preceding or following aspects, or in the alternative to some aspects, the fluid can be a lubrication fluid.

In a fortieth aspect, in addition to one or more of the preceding or following aspects, or in the alternative to some aspects, the fluid can be a hydraulic fluid.

In a forty-first aspect, in addition to one or more of the preceding or following aspects, or in the alternative to some aspects, the fluid can be a gas turbine oil.

In a forty-second aspect, in addition to one or more of the preceding or following aspects, or in the alternative to some aspects, the system for monitoring varnish compounds in fluids can be configured to issue an alert or communication when varnish compounds are detected.

In a forty-third aspect, in addition to one or more of the preceding or following aspects, or in the alternative to some aspects, the system for monitoring varnish compounds in fluids can be configured to initiate varnish compound removal and/or mitigation when varnish compounds are detected.

In a forty-fourth aspect, in addition to one or more of the preceding or following aspects, or in the alternative to some aspects, the system for monitoring varnish compounds in fluids can be configured to detect varnish compound removal and/or remediation after a removal or mitigation operation.

In a forty-fifth aspect, in addition to one or more of the preceding or following aspects, or in the alternative to some aspects, the system for monitoring varnish compounds in fluids can be configured to measure a density of the fluid as an additional property with the fluid property sensor, monitor the density over a time period, and identify an occurrence of a rate of change in the density value indicating a presence of varnish compounds.

In a forty-sixth aspect, in addition to one or more of the preceding or following aspects, or in the alternative to some aspects, the system for monitoring varnish compounds in fluids can be configured to measure a resistivity of the fluid as an additional property with the fluid property sensor, monitor the resistivity over a time period, and identify an occurrence of a rate of change in the resistivity value indicating a presence of varnish compounds.

In a forty-seventh aspect, in addition to one or more of the preceding or following aspects, or in the alternative to some aspects, the system for monitoring varnish compounds in fluids can be configured to measure a viscosity of the fluid as an additional property with the fluid property sensor, monitor the viscosity over a time period, and identify an occurrence of a rate of change in the viscosity value indicating a presence of varnish compounds.

In a forty-eighth aspect, in addition to one or more of the preceding or following aspects, or in the alternative to some aspects, the system for monitoring varnish compounds in fluids can be configured to measure a non-fluid property, monitor the non-fluid property over a time period, and identify an occurrence of a rate of change in the non-fluid property value indicating a presence of varnish compounds.

In a forty-ninth aspect, in addition to one or more of the preceding or following aspects, or in the alternative to some aspects, the non-fluid property includes a differential pressure value of a system filter.

In a fiftieth aspect, in addition to one or more of the preceding or following aspects, or in the alternative to some aspects, the varnish compounds include hard varnish compounds.

In a fifty-first aspect, in addition to one or more of the preceding or following aspects, or in the alternative to some aspects, the varnish compounds include soft varnish compounds.

In a fifty-second aspect, a method of monitoring varnish compounds in fluids can be included. The method can include measuring a dielectric constant of a fluid with a fluid property sensor, monitoring the dielectric constant value over a time period, and predicting a time of the onset of varnish compound formation based on the trend of the dielectric constant value over time.

In a fifty-third aspect, in addition to one or more of the preceding or following aspects, or in the alternative to some aspects, the method can further include estimating an amount of time it will take for the dielectric constant to hit a particular value based on the trend of the dielectric constant value over time.

In a fifty-fourth aspect, in addition to one or more of the preceding or following aspects, or in the alternative to some aspects, the particular value can be predetermined or dynamically determined.

In a fifty-fifth aspect, in addition to one or more of the preceding or following aspects, or in the alternative to some aspects, the method can further include estimating an amount of time it will take for the dielectric constant to hit a particular value based on a comparison of the trend of the dielectric constant value over time against previously stored data regarding how dielectric constant values change over time for a comparable fluid and/or a comparable system in which the fluid resides.

In a fifty-sixth aspect, in addition to one or more of the preceding or following aspects, or in the alternative to some aspects, the method can further include matching the trend of the dielectric constant value over time against a set of previously stored patterns reflecting different time periods until an onset of varnish compound formation begins.

In a fifty-seventh aspect, in addition to one or more of the preceding or following aspects, or in the alternative to some aspects, the method can further include identifying an occurrence of a rate of change in the dielectric constant value indicating an onset of varnish compound formation.

In a fifty-eighth aspect, in addition to one or more of the preceding or following aspects, or in the alternative to some aspects, the method can further include identifying a period of a linear rate of change in the fluid property followed by a period of a non-linear rate of change in the fluid property.

In a fifty-ninth aspect, in addition to one or more of the preceding or following aspects, or in the alternative to some aspects, the method can further include identifying a second period of a linear rate of change in the fluid property following the period of non-linear rate of change.

In a sixtieth aspect, in addition to one or more of the preceding or following aspects, or in the alternative to some aspects, the fluid property can be dielectric constant.

In a sixty-first aspect, in addition to one or more of the preceding or following aspects, or in the alternative to some aspects, the method can further include fitting or modeling the measured dielectric constant value.

In a sixty-second aspect, in addition to one or more of the preceding or following aspects, or in the alternative to some aspects, fitting or modeling the measured dielectric constant value can be performed using at least one of a polynomial, hyperbolic, exponential, logistic, sigmoid, or inverse Carreau-Yasuda equation.

In a sixty-third aspect, in addition to one or more of the preceding or following aspects, or in the alternative to some aspects, the method can further include measuring temperature of the fluid with a temperature sensor.

In a sixty-fourth aspect, in addition to one or more of the preceding or following aspects, or in the alternative to some aspects, the method can further include normalizing the measured dielectric constant value based on the measured fluid temperature.

In a sixty-fifth aspect, in addition to one or more of the preceding or following aspects, or in the alternative to some aspects, the method can further include placing the fluid property sensor after a high heat transfer location in a fluid circuit or at a location with a high differential temperature in a fluid circuit.

In a sixty-sixth aspect, in addition to one or more of the preceding or following aspects, or in the alternative to some aspects, the high heat transfer location in a fluid circuit can be a heat exchanger or cooler in a fluid circuit.

In a sixty-seventh aspect, in addition to one or more of the preceding or following aspects, or in the alternative to some aspects, the fluid property sensor can be a tuning fork-type fluid property sensor.

In a sixty-eighth aspect, in addition to one or more of the preceding or following aspects, or in the alternative to some aspects, the fluid can be a hydrocarbon fluid.

In a sixty-ninth aspect, in addition to one or more of the preceding or following aspects, or in the alternative to some aspects, the fluid can be a lubrication fluid.

In a seventieth aspect, in addition to one or more of the preceding or following aspects, or in the alternative to some aspects, the fluid can be a hydraulic fluid.

In a seventy-first aspect, in addition to one or more of the preceding or following aspects, or in the alternative to some aspects, the fluid can be a gas turbine oil.

In a seventy-second aspect, in addition to one or more of the preceding or following aspects, or in the alternative to some aspects, the method can further include issuing an alert or communication based on the predicted time of the onset of varnish compound formation.

In a seventy-third aspect, in addition to one or more of the preceding or following aspects, or in the alternative to some aspects, the method can further include initiating varnish compound removal and/or mitigation at a particular time based on the predicted time of the onset of varnish compound formation.

In a seventy-fourth aspect, in addition to one or more of the preceding or following aspects, or in the alternative to some aspects, the method can further include detecting varnish compound removal and/or remediation after a removal or mitigation operation.

In a seventy-fifth aspect, in addition to one or more of the preceding or following aspects, or in the alternative to some aspects, methods herein can further include measuring a density of the fluid as an additional property with the fluid property sensor, monitoring the density over a time period, and predicting a time of the onset of varnish compound formation based on the trend of the density value over time.

In a seventy-sixth aspect, in addition to one or more of the preceding or following aspects, or in the alternative to some aspects, methods herein can further include measuring a resistivity of the fluid as an additional property with the fluid property sensor, monitoring the resistivity over a time period, and predicting a time of the onset of varnish compound formation based on the trend of the resistivity value over time.

In a seventy-seventh aspect, in addition to one or more of the preceding or following aspects, or in the alternative to some aspects, methods herein can further include measuring a viscosity of the fluid as an additional property with the fluid property sensor, monitoring the viscosity over a time period, and predicting a time of the onset of varnish compound formation based on the trend of the viscosity value over time.

In a seventy-eighth aspect, in addition to one or more of the preceding or following aspects, or in the alternative to some aspects, methods herein can further include measuring a non-fluid property, monitoring the non-fluid property over a time period, and predicting a time of the onset of varnish compound formation based on the trend of the non-fluid property value over time.

In a seventy-ninth aspect, in addition to one or more of the preceding or following aspects, or in the alternative to some aspects, the varnish compounds include hard varnish compounds.

In an eightieth aspect, in addition to one or more of the preceding or following aspects, or in the alternative to some aspects, the varnish compounds include soft varnish compounds.

In an eighty-first aspect, a system for monitoring varnish compounds in fluids can be included having a control circuit and a fluid property sensor. The system for monitoring varnish compounds in fluids can be configured to measure a dielectric constant of a fluid with the fluid property sensor, monitor the dielectric constant over a time period, and predict a time of the onset of varnish compound formation based on the trend of the dielectric constant value over time.

In an eighty-second aspect, in addition to one or more of the preceding or following aspects, or in the alternative to some aspects, the system for monitoring varnish compounds in fluids can be configured to estimate an amount of time it will take for the dielectric constant to hit a particular value based on the trend of the dielectric constant value over time.

In an eighty-third aspect, in addition to one or more of the preceding or following aspects, or in the alternative to some aspects, the particular value can be predetermined or dynamically determined.

In an eighty-fourth aspect, in addition to one or more of the preceding or following aspects, or in the alternative to some aspects, the system for monitoring varnish compounds in fluids can be configured to estimate an amount of time it will take for the dielectric constant to hit a particular value based on a comparison of the trend of the dielectric constant value over time against previously stored data regarding how dielectric constant values change over time for a comparable fluid and/or a comparable system in which the fluid resides.

In an eighty-fifth aspect, in addition to one or more of the preceding or following aspects, or in the alternative to some aspects, the system for monitoring varnish compounds in fluids can be configured to match the trend of the dielectric constant value over time against a set of previously stored patterns reflecting different time periods until an onset of varnish compound formation begins.

In an eighty-sixth aspect, in addition to one or more of the preceding or following aspects, or in the alternative to some aspects, the system for monitoring varnish compounds in fluids can be configured to identify an occurrence of the rate of change in the dielectric constant value indicating an onset of varnish compound formation.

In an eighty-seventh aspect, in addition to one or more of the preceding or following aspects, or in the alternative to some aspects, the system for monitoring varnish compounds in fluids can be configured to identify a period of a linear rate of change in the fluid property followed by a period of a non-linear rate of change in the fluid property.

In an eighty-eighth aspect, in addition to one or more of the preceding or following aspects, or in the alternative to some aspects, the system for monitoring varnish compounds in fluids can be configured to identify a second period of a linear rate of change in the fluid property following the period of non-linear rate of change.

In an eighty-ninth aspect, in addition to one or more of the preceding or following aspects, or in the alternative to some aspects, the system for monitoring varnish compounds in fluids can be configured to fit or model the measured dielectric constant value.

In a ninetieth aspect, in addition to one or more of the preceding or following aspects, or in the alternative to some aspects, wherein fitting or modeling the measured dielectric constant value can be performed using at least one of a polynomial, hyperbolic, exponential, logistic, sigmoid, or an inverse Carreau-Yasuda equation.

In a ninety-first aspect, in addition to one or more of the preceding or following aspects, or in the alternative to some aspects, can further include a temperature sensor, wherein the system for monitoring varnish compounds in fluids can be configured to measure a temperature of the fluid using the temperature sensor.

In a ninety-second aspect, in addition to one or more of the preceding or following aspects, or in the alternative to some aspects, the system for monitoring varnish compounds in fluids can be configured to normalize the measured dielectric constant value based on the measured fluid temperature.

In a ninety-third aspect, in addition to one or more of the preceding or following aspects, or in the alternative to some aspects, the fluid property sensor can be positioned after a high heat transfer location in a fluid circuit or at a location with a high differential temperature in a fluid circuit.

In a ninety-fourth aspect, in addition to one or more of the preceding or following aspects, or in the alternative to some aspects, the high heat transfer location can be a heat exchanger or cooler in a fluid circuit.

In a ninety-fifth aspect, in addition to one or more of the preceding or following aspects, or in the alternative to some aspects, the fluid property sensor can be a tuning fork-type fluid property sensor.

In a ninety-sixth aspect, in addition to one or more of the preceding or following aspects, or in the alternative to some aspects, the fluid can be a hydrocarbon fluid.

In a ninety-seventh aspect, in addition to one or more of the preceding or following aspects, or in the alternative to some aspects, the fluid can be a lubrication fluid.

In a ninety-eighth aspect, in addition to one or more of the preceding or following aspects, or in the alternative to some aspects, the fluid can be a hydraulic fluid.

In a ninety-ninth aspect, in addition to one or more of the preceding or following aspects, or in the alternative to some aspects, the fluid can be a gas turbine oil.

In a one hundredth aspect, in addition to one or more of the preceding or following aspects, or in the alternative to some aspects, the system for monitoring varnish compounds in fluids can be configured to issue an alert or communication based on the predicted time of the onset of varnish compound formation.

In a one hundred and first aspect, in addition to one or more of the preceding or following aspects, or in the alternative to some aspects, the system for monitoring varnish compounds in fluids can be configured to initiate varnish compound removal and/or mitigation at a particular time based on the predicted time of the onset of varnish compound formation.

In a one hundred and second aspect, in addition to one or more of the preceding or following aspects, or in the alternative to some aspects, the system for monitoring varnish compounds in fluids can be configured to detect varnish compound removal and/or remediation after a removal or mitigation operation.

In a one hundred and third aspect, in addition to one or more of the preceding or following aspects, or in the alternative to some aspects, the system for monitoring varnish compounds in fluids can be configured to measure a density of the fluid as an additional property with the fluid property sensor, monitor the density over a time period, and predict a time of the onset of varnish compound formation based on the trend of the density value over time.

In a one hundred and fourth aspect, in addition to one or more of the preceding or following aspects, or in the alternative to some aspects, the system for monitoring varnish compounds in fluids can be configured to measure a resistivity of the fluid as an additional property with the fluid property sensor, monitor the resistivity over a time period, and predict a time of the onset of varnish compound formation based on the trend of the resistivity value over time.

In a one hundred and fifth aspect, in addition to one or more of the preceding or following aspects, or in the alternative to some aspects, the system for monitoring varnish compounds in fluids can be configured to measure a viscosity of the fluid as an additional property with the fluid property sensor, monitor the viscosity over a time period, and predict a time of the onset of varnish compound formation based on the trend of the viscosity value over time.

In a one hundred and sixth aspect, in addition to one or more of the preceding or following aspects, or in the alternative to some aspects, the system for monitoring varnish compounds in fluids can be configured to measure a non-fluid property, monitor the non-fluid property over a time period, and predict a time of the onset of varnish compound formation based on the trend of the non-fluid property value over time.

In a one hundred and seventh aspect, in addition to one or more of the preceding or following aspects, or in the alternative to some aspects, the non-fluid property includes a differential pressure value of a system filter.

In a one hundred and eighth aspect, in addition to one or more of the preceding or following aspects, or in the alternative to some aspects, the varnish compounds include hard varnish compounds.

In a one hundred and ninth aspect, in addition to one or more of the preceding or following aspects, or in the alternative to some aspects, the varnish compounds include soft varnish compounds.

In a one hundred and tenth aspect, a method of monitoring varnish compounds in fluids can be included. The method can include measuring a density of a fluid with a fluid property sensor, monitoring the density over a time period, and identifying an occurrence of a rate of change in the density value indicating a presence of varnish compounds.

In a one hundred and eleventh aspect, in addition to one or more of the preceding or following aspects, or in the alternative to some aspects, the fluid property sensor can be a tuning fork-type fluid property sensor.

In a one hundred and twelfth aspect, in addition to one or more of the preceding or following aspects, or in the alternative to some aspects, the method can further include identifying an occurrence of the rate of change in the density value indicating an onset of varnish compound formation.

In a one hundred and thirteenth aspect, in addition to one or more of the preceding or following aspects, or in the alternative to some aspects, the method can further include identifying a period of a linear rate of change in the fluid property followed by a period of a non-linear rate of change in the fluid property.

In a one hundred and fourteenth aspect, in addition to one or more of the preceding or following aspects, or in the alternative to some aspects, the method can further include identifying a second period of a linear rate of change in the fluid property following the period of non-linear rate of change.

In a one hundred and fifteenth aspect, in addition to one or more of the preceding or following aspects, or in the alternative to some aspects, the fluid property can be density.

In a one hundred and sixteenth aspect, in addition to one or more of the preceding or following aspects, or in the alternative to some aspects, the method can further include measuring temperature of the fluid with a temperature sensor.

In a one hundred and seventeenth aspect, in addition to one or more of the preceding or following aspects, or in the alternative to some aspects, the method can further include normalizing the measured density value based on the measured fluid temperature.

In a one hundred and eighteenth aspect, in addition to one or more of the preceding or following aspects, or in the alternative to some aspects, the method can further include placing the fluid property sensor after a high heat transfer location in a fluid circuit or at a location with a high differential temperature in a fluid circuit.

In a one hundred and nineteenth aspect, in addition to one or more of the preceding or following aspects, or in the alternative to some aspects, the high heat transfer location in a fluid circuit can be a heat exchanger or cooler in a fluid circuit.

In a one hundred and twentieth aspect, in addition to one or more of the preceding or following aspects, or in the alternative to some aspects, the fluid can be a hydrocarbon fluid.

In a one hundred and twenty-first aspect, in addition to one or more of the preceding or following aspects, or in the alternative to some aspects, the fluid can be a lubrication fluid.

In a one hundred and twenty-second aspect, in addition to one or more of the preceding or following aspects, or in the alternative to some aspects, the fluid can be a hydraulic fluid.

In a one hundred and twenty-third aspect, in addition to one or more of the preceding or following aspects, or in the alternative to some aspects, the fluid can be a gas turbine oil.

In a one hundred and twenty-fourth aspect, in addition to one or more of the preceding or following aspects, or in the alternative to some aspects, the method can further include issuing an alert or communication when varnish compounds are detected.

In a one hundred and twenty-fifth aspect, in addition to one or more of the preceding or following aspects, or in the alternative to some aspects, the method can further include initiating varnish compound removal and/or mitigation when varnish compounds are detected.

In a one hundred and twenty-sixth aspect, in addition to one or more of the preceding or following aspects, or in the alternative to some aspects, the method can further include identifying when a measured fluid density value exceeds that of water.

In a one hundred and twenty-seventh aspect, in addition to one or more of the preceding or following aspects, or in the alternative to some aspects, methods herein can further include measuring a dielectric constant of the fluid with the fluid property sensor, monitoring the dielectric constant over a time period, and identifying an occurrence of a rate of change in the dielectric constant value indicating a presence of varnish compounds.

In a one hundred and twenty-eighth aspect, in addition to one or more of the preceding or following aspects, or in the alternative to some aspects, methods herein can further include measuring a resistivity of the fluid with the fluid property sensor, monitoring the resistivity over a time period, and identifying an occurrence of a rate of change in the resistivity value indicating a presence of varnish compounds.

In a one hundred and twenty-ninth aspect, in addition to one or more of the preceding or following aspects, or in the alternative to some aspects, methods herein can further include measuring a viscosity of the fluid with the fluid property sensor, monitoring the viscosity over a time period, and identifying an occurrence of a rate of change in the viscosity value indicating a presence of varnish compounds.

In a one hundred and thirtieth aspect, in addition to one or more of the preceding or following aspects, or in the alternative to some aspects, methods herein can further include measuring a non-fluid property, monitoring the non-fluid property over a time period, and predicting a time of the onset of varnish compound formation based on the trend of the non-fluid property value over time.

In a one hundred and thirty-first aspect, in addition to one or more of the preceding or following aspects, or in the alternative to some aspects, the varnish compounds include hard varnish compounds.

In a one hundred and thirty-second aspect, in addition to one or more of the preceding or following aspects, or in the alternative to some aspects, the varnish compounds include soft varnish compounds.

In a one hundred and thirty-third aspect, a method of monitoring varnish compounds in fluids can be included, the method including measuring a density of a fluid with a fluid property sensor, monitoring the density value over a time period, and predicting a time of the onset of varnish compound formation based on the trend of the density value over time.

In a one hundred and thirty-fourth aspect, in addition to one or more of the preceding or following aspects, or in the alternative to some aspects, the method can further include estimating an amount of time it will take for the density to hit a particular value based on the trend of the density value over time.

In a one hundred and thirty-fifth aspect, in addition to one or more of the preceding or following aspects, or in the alternative to some aspects, the particular value can be predetermined or dynamically determined.

In a one hundred and thirty-sixth aspect, in addition to one or more of the preceding or following aspects, or in the alternative to some aspects, the method can further include estimating an amount of time it will take for the density to hit a particular value based on a comparison of the trend of the density over time against previously stored data regarding how density values change over time for a comparable fluid and/or a comparable system in which the fluid resides.

In a one hundred and thirty-seventh aspect, in addition to one or more of the preceding or following aspects, or in the alternative to some aspects, the method can further include matching the trend of the density value over time against a set of previously stored patterns reflecting different time periods until an onset of varnish compound formation begins.

In a one hundred and thirty-eighth aspect, in addition to one or more of the preceding or following aspects, or in the alternative to some aspects, the method can further include identifying an occurrence of a rate of change in the density value indicating an onset of varnish compound formation.

In a one hundred and thirty-ninth aspect, in addition to one or more of the preceding or following aspects, or in the alternative to some aspects, the method of monitoring varnish compounds in fluids can be configured to identify a period of a linear rate of change in the fluid property followed by a period of a non-linear rate of change in the fluid property.

In a one hundred and fortieth aspect, in addition to one or more of the preceding or following aspects, or in the alternative to some aspects, the method of monitoring varnish compounds in fluids can be configured to identify a second period of a linear rate of change in the fluid property following the period of non-linear rate of change.

In a one hundred and forty-first aspect, in addition to one or more of the preceding or following aspects, or in the alternative to some aspects, the method can further include measuring temperature of the fluid with a temperature sensor.

In a one hundred and forty-second aspect, in addition to one or more of the preceding or following aspects, or in the alternative to some aspects, the method can further include normalizing the measured density value based on the measured fluid temperature.

In a one hundred and forty-third aspect, in addition to one or more of the preceding or following aspects, or in the alternative to some aspects, the method can further include placing the fluid property sensor after a high heat transfer location in a fluid circuit or at a location with a high differential temperature in a fluid circuit.

In a one hundred and forty-fourth aspect, in addition to one or more of the preceding or following aspects, or in the alternative to some aspects, the fluid property sensor can be a tuning fork-type fluid property sensor.

In a one hundred and forty-fifth aspect, in addition to one or more of the preceding or following aspects, or in the alternative to some aspects, the fluid can be a hydrocarbon fluid.

In a one hundred and forty-sixth aspect, in addition to one or more of the preceding or following aspects, or in the alternative to some aspects, the fluid can be a lubrication fluid.

In a one hundred and forty-seventh aspect, in addition to one or more of the preceding or following aspects, or in the alternative to some aspects, the fluid can be a hydraulic fluid.

In a one hundred and forty-eighth aspect, in addition to one or more of the preceding or following aspects, or in the alternative to some aspects, the fluid can be a gas turbine oil.

In a one hundred and forty-ninth aspect, in addition to one or more of the preceding or following aspects, or in the alternative to some aspects, the method can further include issuing an alert or communication based on the predicted time of the onset of varnish compound formation.

In a one hundred and fiftieth aspect, in addition to one or more of the preceding or following aspects, or in the alternative to some aspects, the method can further include initiating varnish compound removal and/or mitigation at a particular time based on the predicted time of the onset of varnish compound formation.

In a one hundred and fifty-first aspect, in addition to one or more of the preceding or following aspects, or in the alternative to some aspects, the method can further include detecting varnish compound removal and/or remediation after a removal or mitigation operation.

In a one hundred and fifty-second aspect, in addition to one or more of the preceding or following aspects, or in the alternative to some aspects, methods herein can further include measuring a resistivity of the fluid with the fluid property sensor, monitoring the resistivity over a time period, and predicting a time of the onset of varnish compound formation based on the trend of the resistivity value over time.

In a one hundred and fifty-third aspect, in addition to one or more of the preceding or following aspects, or in the alternative to some aspects, methods herein can further include measuring a dielectric constant of the fluid with the fluid property sensor, monitoring the dielectric constant over a time period, and predicting a time of the onset of varnish compound formation based on the trend of the dielectric constant value over time.

In a one hundred and fifty-fourth aspect, in addition to one or more of the preceding or following aspects, or in the alternative to some aspects, methods herein can further include measuring a viscosity of the fluid with the fluid property sensor, monitoring the viscosity over a time period, and predicting a time of the onset of varnish compound formation based on the trend of the viscosity value over time.

In a one hundred and fifty-fifth aspect, in addition to one or more of the preceding or following aspects, or in the alternative to some aspects, methods herein can further include measuring a non-fluid property, monitoring the non-fluid property over a time period, and predicting a time of the onset of varnish compound formation based on the trend of the non-fluid property value over time.

In a one hundred and fifty-sixth aspect, in addition to one or more of the preceding or following aspects, or in the alternative to some aspects, the varnish compounds include hard varnish compounds.

In a one hundred and fifty-seventh aspect, in addition to one or more of the preceding or following aspects, or in the alternative to some aspects, the varnish compounds include soft varnish compounds.

In a one hundred and fifty-eighth aspect, a method of monitoring varnish compounds in fluids can be included, the method including measuring a resistivity of a fluid with a fluid property sensor, monitoring the resistivity over a time period, and identifying an occurrence of a rate of change in the resistivity value indicating a presence of varnish compounds.

In a one hundred and fifty-ninth aspect, in addition to one or more of the preceding or following aspects, or in the alternative to some aspects, the fluid property sensor can be a tuning fork-type fluid property sensor.

In a one hundred and sixtieth aspect, in addition to one or more of the preceding or following aspects, or in the alternative to some aspects, the method can further include identifying an occurrence of the rate of change in the resistivity value indicating an onset of varnish compound formation.

In a one hundred and sixty-first aspect, in addition to one or more of the preceding or following aspects, or in the alternative to some aspects, the method can further include identifying a period of a linear rate of change in the fluid property followed by a period of a non-linear rate of change in the fluid property.

In a one hundred and sixty-second aspect, in addition to one or more of the preceding or following aspects, or in the alternative to some aspects, the method can further include identifying a second period of a linear rate of change in the fluid property following the period of non-linear rate of change.

In a one hundred and sixty-third aspect, in addition to one or more of the preceding or following aspects, or in the alternative to some aspects, the fluid property can be resistivity.

In a one hundred and sixty-fourth aspect, in addition to one or more of the preceding or following aspects, or in the alternative to some aspects, the method can further include measuring temperature of the fluid with a temperature sensor.

In a one hundred and sixty-fifth aspect, in addition to one or more of the preceding or following aspects, or in the alternative to some aspects, the method can further include normalizing the measured resistivity value based on the measured fluid temperature.

In a one hundred and sixty-sixth aspect, in addition to one or more of the preceding or following aspects, or in the alternative to some aspects, the method can further include placing the fluid property sensor after a high heat transfer location in a fluid circuit or at a location with a high differential temperature in a fluid circuit.

In a one hundred and sixty-seventh aspect, in addition to one or more of the preceding or following aspects, or in the alternative to some aspects, the high heat transfer location in a fluid circuit can be a heat exchanger or cooler in a fluid circuit.

In a one hundred and sixty-eighth aspect, in addition to one or more of the preceding or following aspects, or in the alternative to some aspects, the fluid can be a hydrocarbon fluid.

In a one hundred and sixty-ninth aspect, in addition to one or more of the preceding or following aspects, or in the alternative to some aspects, the fluid can be a lubrication fluid.

In a one hundred and seventieth aspect, in addition to one or more of the preceding or following aspects, or in the alternative to some aspects, the fluid can be a hydraulic fluid.

In a one hundred and seventy-first aspect, in addition to one or more of the preceding or following aspects, or in the alternative to some aspects, the fluid can be a gas turbine oil.

In a one hundred and seventy-second aspect, in addition to one or more of the preceding or following aspects, or in the alternative to some aspects, the method can further include issuing an alert or communication when varnish compounds are detected.

In a one hundred and seventy-third aspect, in addition to one or more of the preceding or following aspects, or in the alternative to some aspects, the method can further include initiating varnish compound removal and/or mitigation when varnish compounds are detected.

In a one hundred and seventy-fourth aspect, in addition to one or more of the preceding or following aspects, or in the alternative to some aspects, the method can further include identifying when a measured fluid density value exceeds that of water.

In a one hundred and seventy-fifth aspect, in addition to one or more of the preceding or following aspects, or in the alternative to some aspects, methods herein can further include measuring a dielectric constant of the fluid with the fluid property sensor, monitoring the dielectric constant over a time period, and identifying an occurrence of a rate of change in the dielectric constant value indicating a presence of varnish compounds.

In a one hundred and seventy-sixth aspect, in addition to one or more of the preceding or following aspects, or in the alternative to some aspects, methods herein can further include measuring a density of the fluid with the fluid property sensor, monitoring the density over a time period, and identifying an occurrence of a rate of change in the density value indicating a presence of varnish compounds.

In a one hundred and seventy-seventh aspect, in addition to one or more of the preceding or following aspects, or in the alternative to some aspects, methods herein can further include measuring a viscosity of the fluid with the fluid property sensor, monitoring the viscosity over a time period, and identifying an occurrence of a rate of change in the viscosity value indicating a presence of varnish compounds.

In a one hundred and seventy-eighth aspect, in addition to one or more of the preceding or following aspects, or in the alternative to some aspects, methods herein can further include measuring a non-fluid property, monitoring the non-fluid property over a time period, and predicting a time of the onset of varnish compound formation based on the trend of the non-fluid property value over time.

In a one hundred and seventy-ninth aspect, in addition to one or more of the preceding or following aspects, or in the alternative to some aspects, the varnish compounds include hard varnish compounds.

In a one hundred and eightieth aspect, in addition to one or more of the preceding or following aspects, or in the alternative to some aspects, the varnish compounds include soft varnish compounds.

In a one hundred and eighty-first aspect, a method of monitoring varnish compounds in fluids can be included, the method including measuring a resistivity of a fluid with a fluid property sensor, monitoring the resistivity value over a time period, and predicting a time of the onset of varnish compound formation based on the trend of the resistivity value over time.

In a one hundred and eighty-second aspect, in addition to one or more of the preceding or following aspects, or in the alternative to some aspects, the method can further include estimating an amount of time it will take for the density to hit a particular value based on the trend of the resistivity value over time.

In a one hundred and eighty-third aspect, in addition to one or more of the preceding or following aspects, or in the alternative to some aspects, the particular value can be predetermined or dynamically determined.

In a one hundred and eighty-fourth aspect, in addition to one or more of the preceding or following aspects, or in the alternative to some aspects, the method can further include estimating an amount of time it will take for the resistivity to hit a particular value based on a comparison of the trend of the resistivity over time against previously stored data regarding how density values change over time for a comparable fluid and/or a comparable system in which the fluid resides.

In a one hundred and eighty-fifth aspect, in addition to one or more of the preceding or following aspects, or in the alternative to some aspects, the method can further include matching the trend of the resistivity value over time against a set of previously stored patterns reflecting different time periods until an onset of varnish compound formation begins.

In a one hundred and eighty-sixth aspect, in addition to one or more of the preceding or following aspects, or in the alternative to some aspects, the method can further include identifying an occurrence of a rate of change in the resistivity value indicating an onset of varnish compound formation.

In a one hundred and eighty-seventh aspect, in addition to one or more of the preceding or following aspects, or in the alternative to some aspects, the method of monitoring varnish compounds in fluids can be configured to identify a period of a linear rate of change in the fluid property followed by a period of a non-linear rate of change in the fluid property.

In a one hundred and eighty-eighth aspect, in addition to one or more of the preceding or following aspects, or in the alternative to some aspects, the method of monitoring varnish compounds in fluids can be configured to identify a second period of a linear rate of change in the fluid property following the period of non-linear rate of change.

In a one hundred and eighty-ninth aspect, in addition to one or more of the preceding or following aspects, or in the alternative to some aspects, the method can further include measuring temperature of the fluid with a temperature sensor.

In a one hundred and ninetieth aspect, in addition to one or more of the preceding or following aspects, or in the alternative to some aspects, the method can further include normalizing the measured resistivity value based on the measured fluid temperature.

In a one hundred and ninety-first aspect, in addition to one or more of the preceding or following aspects, or in the alternative to some aspects, the method can further include placing the fluid property sensor after a high heat transfer location in a fluid circuit or at a location with a high differential temperature in a fluid circuit.

In a one hundred and ninety-second aspect, in addition to one or more of the preceding or following aspects, or in the alternative to some aspects, the fluid property sensor can be a tuning fork-type fluid property sensor.

In a one hundred and ninety-third aspect, in addition to one or more of the preceding or following aspects, or in the alternative to some aspects, the fluid can be a hydrocarbon fluid.

In a one hundred and ninety-fourth aspect, in addition to one or more of the preceding or following aspects, or in the alternative to some aspects, the fluid can be a lubrication fluid.

In a one hundred and ninety-fifth aspect, in addition to one or more of the preceding or following aspects, or in the alternative to some aspects, the fluid can be a hydraulic fluid.

In a one hundred and ninety-sixth aspect, in addition to one or more of the preceding or following aspects, or in the alternative to some aspects, the fluid can be a gas turbine oil.

In a one hundred and ninety-seventh aspect, in addition to one or more of the preceding or following aspects, or in the alternative to some aspects, the method can further include issuing an alert or communication based on the predicted time of the onset of varnish compound formation.

In a one hundred and ninety-eighth aspect, in addition to one or more of the preceding or following aspects, or in the alternative to some aspects, the method can further include initiating varnish compound removal and/or mitigation at a particular time based on the predicted time of the onset of varnish compound formation.

In a one hundred and ninety-ninth aspect, in addition to one or more of the preceding or following aspects, or in the alternative to some aspects, the method can further include detecting varnish compound removal and/or remediation after a removal or mitigation operation.

In a two hundredth aspect, in addition to one or more of the preceding or following aspects, or in the alternative to some aspects, methods herein can further include measuring a dielectric constant of the fluid with the fluid property sensor, monitoring the dielectric constant over a time period, and predicting a time of the onset of varnish compound formation based on the trend of the dielectric constant value over time.

In a two hundred and first aspect, in addition to one or more of the preceding or following aspects, or in the alternative to some aspects, methods herein can further include measuring a density of the fluid with the fluid property sensor, monitoring the density over a time period, and predicting a time of the onset of varnish compound formation based on the trend of the density value over time.

In a two hundred and second aspect, in addition to one or more of the preceding or following aspects, or in the alternative to some aspects, methods herein can further include measuring a viscosity of the fluid with the fluid property sensor, monitoring the viscosity over a time period, and predicting a time of the onset of varnish compound formation based on the trend of the viscosity value over time.

In a two hundred and third aspect, in addition to one or more of the preceding or following aspects, or in the alternative to some aspects, methods herein can further include measuring a non-fluid property, monitoring the non-fluid property over a time period, and predicting a time of the onset of varnish compound formation based on the trend of the non-fluid property value over time.

In a two hundred and fourth aspect, in addition to one or more of the preceding or following aspects, or in the alternative to some aspects, the varnish compounds include hard varnish compounds.

In a two hundred and fifth aspect, in addition to one or more of the preceding or following aspects, or in the alternative to some aspects, the varnish compounds include soft varnish compounds.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of a piece of equipment including a monitoring system in accordance with various embodiments herein.

FIG. 2 is a schematic view of a hydraulic system for monitoring in accordance with various embodiments herein.

FIG. 3 is a schematic view of components of a system for varnish detection and/or monitoring herein.

FIG. 4 shows a set of charts illustrating a change in density of an oil over time as varnish compounds are formed.

FIG. 5 shows a set of charts illustrating a change in dielectric constant of an oil over time as varnish compounds are formed.

FIG. 6 is a chart showing a fluid property value over time exhibiting linear and non-linear rates of change.

FIG. 7 is a block diagram view of components of system for varnish detection and/or monitoring in accordance with various embodiments herein.

FIG. 8 is a flow chart of operations in accordance with various embodiments herein.

FIG. 9 is a flow chart of operations in accordance with various embodiments herein.

FIG. 10 is a flow chart of operations in accordance with various embodiments herein.

FIG. 11 is a flow chart of operations in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

As referenced above, varnish compounds represent a contaminant causing harmful effects on the systems they become deposited in. Currently, the only way to evaluate fluids for varnish compounds is through off-line testing methods (e.g., analysis in a laboratory). That greatly limits the ability to gather and use such information.

However, embodiments herein can be used to detect and/or monitor for varnish compounds allowing appropriate service to be performed (changing fluids, treating fluids, performing system maintenance, and the like) to mitigate the same before varnish compounds cause substantial problems. Notably, embodiments herein can be used to detect and/or monitor for varnish compounds in-line because analysis can take place on-vehicle or on-equipment including while the system or process is operating (on-line) and not in a laboratory setting.

In various embodiments, a system for monitoring varnish compounds in fluids is included herein including a control circuit in communication with a fluid property sensor. The system can be configured to measure one or more fluid properties (such as dielectric constant, density, resistivity, viscosity and the like) with the fluid property sensor, monitor one or more of the measured properties over a time period, and then identify an occurrence of a rate of change in the measured property indicating a presence of the varnish compounds. In some embodiments, an onset of varnish compound formation can even be predicted. In various embodiments, a system herein can be configured to measure a fluid property of a fluid with a fluid property sensor, monitor the fluid property value over a time period, and predict a time of the onset of varnish compound formation based on the trend of the fluid property value over time. Systems herein can also use such information to determine aspects such as remaining filter life, recommended system servicing times, and the like.

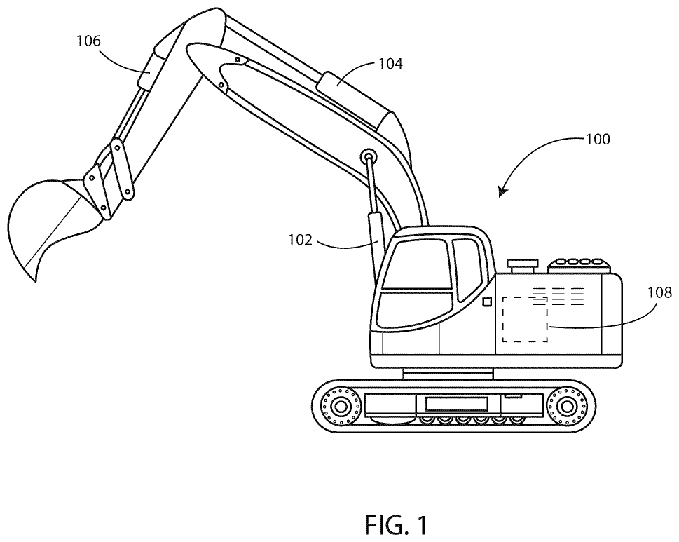

Referring now to FIG. 1, a schematic view is shown of an example piece of equipment 100, which in this example is construction equipment, but could be any type of equipment including, but not limited to, any type of construction equipment, agricultural equipment, transportation equipment, industrial equipment, power generation equipment, manufacturing equipment, or the like. The equipment 100 includes hydraulic actuators including, for example, hydraulic actuators 102, 104, and 106, which depend upon hydraulic fluid for operation. Hydraulic fluid is typically a hydrocarbon fluid and varnish compounds can form therein through thermal and/or oxidative breakdown. In this example, the equipment 100 can also include a varnish detection/monitoring system 108 in accordance with various embodiments herein to detect and/or monitor varnish compounds, or even predict the onset of formation of the same. While the system 108 in FIG. 1 is described with respect to detecting/monitoring/predicting varnish specifically in hydraulic fluids, it will be appreciated that systems herein can also be used to detect/monitor varnish formation in other types of fluids beyond hydraulic fluid including, but not limited to, hydrocarbon liquids of all types including such as fuel, oil, lubricants, coolants, and the like.

In various embodiments, the varnish detection/monitoring system 108 can be configured to record and/or evaluate signals from one or more fluid property sensors, described in greater detail below. For example, the varnish detection/monitoring system 108 can be configured to record and/or evaluate varnish compound formation including dielectric constant, density, resistivity, viscosity, and/or combinations thereof to identify patterns in the fluid property sensor signals associated with the presence of and/or the formation of varnish compounds. In various embodiments, the varnish detection/monitoring system 108 can be configured to specifically identify changes in signal patterns of the fluid property sensor signals associated with the presence of varnish compound formation. In various embodiments, the varnish detection/monitoring system 108 can be configured to predict the onset of varnish compound formation based on tracked changes in fluid properties over time. In various embodiments, the varnish detection/monitoring system 108 can also use data inputs from one or more secondary sensors and, in some cases, sensors that are not fluid property sensors.

Varnish can impact filter performance and/or filter life in fluid filtration systems. In various embodiments, the varnish detection/monitoring system 108 can be configured to calculate and/or estimate various aspects regarding a filtration system or filter element thereof. For example, in various embodiments, the varnish detection/monitoring system 108 can be configured to estimate remaining useful filter life based in part on information related to varnish including the presence of varnish, amounts of the same, the timing of varnish formation onset, amount of time that that fluids have been in use, cumulative heat exposure for the fluids, and the like. In various embodiments, the varnish detection/monitoring system 108 can be configured to estimate filter remaining useful life based on information related to varnish and one or more other parameters including, for example, filter pressure drop, temperature, filter time in-use, volume of fluid filtered, and the like.

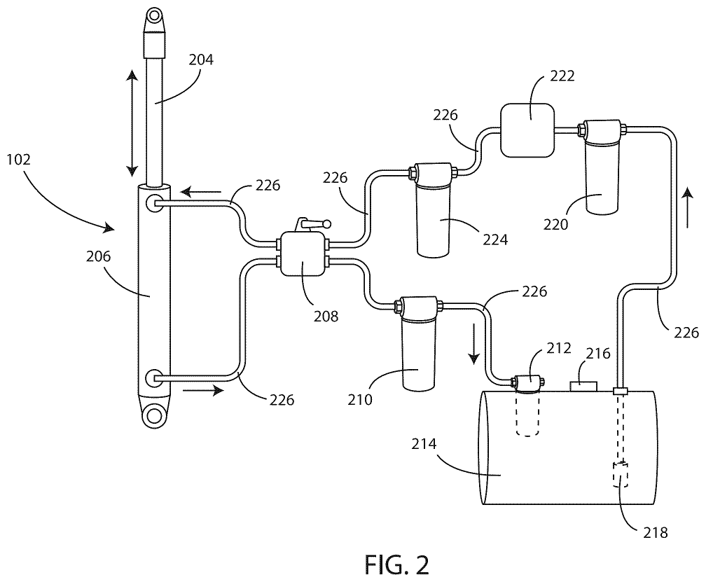

Referring now to FIG. 2, a schematic view of a hydraulic system is shown in accordance with various embodiments herein. It will be appreciated that in many embodiments of hydraulic systems not all of the various filters or other components depicted in FIG. 2 may actually be present. In this example, the illustrated system includes hydraulic actuator 102. The hydraulic actuator 102 includes a cylinder barrel 206 and a piston rod 204. Hydraulic fluid moves through the system 200 as controlled with a control unit 208 and passes through a hydraulic fluid line 226. An amount of hydraulic fluid is stored within a reservoir tank 214 and passes through a strainer 218 before traveling through the hydraulic fluid line 226 and passing to a low (or suction) pressure filter 220 before going to a hydraulic fluid pump 222. The hydraulic fluid is then pumped to a medium or high-pressure filter 224 and then passes through the control unit 208 and then onto the hydraulic actuator 102. On the return path, the hydraulic fluid then passes through the control unit 208 and then passes through a return line filter 210 before passing through an in-tank return filter 212 and entering the reservoir tank 214. The reservoir tank 214 can include a breather 216. In some embodiments, the hydraulic system 200 can also include a kidney loop system (not shown in this view). A kidney loop system can include a pump and a filter and can operate to pump fluid from the reservoir tank 214 through the filter and back to the reservoir tank 214 so that the kidney loop functions to clean the fluid within the reservoir tank 214.

Sensors for varnish detection and/or monitoring herein can be mounted at various points along the hydraulic system 200. In some embodiments, sensors can be mounted along the hydraulic fluid line 226. In some embodiments, sensors can be mounted in areas of a system conducive for the formation of varnish compounds. For example, warmer fluid can hold more varnish compounds in solution, but as fluid cools the largely polar varnish compounds can come out of solution to form deposits. As such, in some embodiments, one or more sensors can be mounted in an area where the fluid cools or is cooler in temperature. For example, in some embodiments, a fluid property sensor herein can be positioned to be after a high heat transfer location in a fluid circuit, such as after a heat exchanger in a fluid circuit or after a cooler in a fluid circuit (such as a fan-type “cooler” or the like).

In some embodiments, fluid property sensors herein can be positioned to be near components that are particularly susceptible to damage from varnish deposits. In some embodiments, fluid property sensors herein can be positioned to be near components that are particularly expensive to replace after damage from varnish deposits. However, it will be appreciated that sensors for systems herein (or components of the same) can be mounted anywhere within a fluid system including, for example, upstream, downstream, on, or in of any of the fluid filters described herein or at other locations. In various embodiments, at least some of the sensors of the systems herein can be mounted on or in fluid flow lines, on or in a filter head, on or in a filter housing, on or in a filter element, or the like.

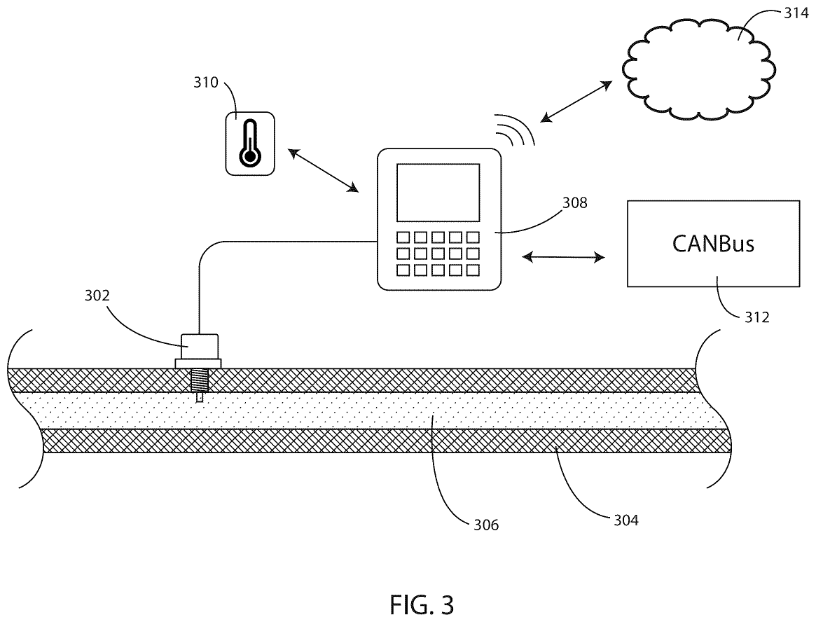

Referring now to FIG. 3 a schematic view of a varnish detection/monitoring and/or prediction system is shown in accordance with various embodiments herein. The varnish detection and/or monitoring system includes a control unit 308. The varnish detection and/or monitoring system also includes a fluid property sensor unit 302 and a secondary sensor unit 310. It will be appreciated that some properties, such as temperature, can be sensed either as part of the fluid property sensor unit 302 or the secondary sensor unit 310, or both, such as temperature. The fluid property sensor unit 302 can be positioned so as to be able to measure a fluid property of a fluid 306 within a fluid line 304. The varnish detection and/or monitoring system can also include a CANBus interface 312 (or another type of data interface) to exchange data with the system in which the fluid is a part. In various embodiments, the control unit 308 can also exchange data with the cloud 314 and/or various remote computing resources. Various processing steps can be performed herein with some being performed locally on the control unit 308, some being performed in the cloud 314 and/or on a remote computing resource, and some being performed in multiple locations.

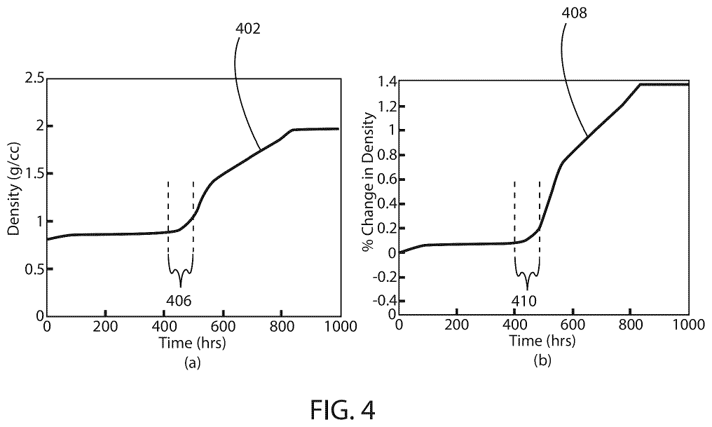

The fluid property sensor unit (and/or one or more secondary sensors) can detect fluid properties and in some cases non-fluid properties, that can be used to detect varnish, monitor the same, and/or predict the onset of varnish compound formation. Varnish compounds herein can include both soluble and insoluble varnish compounds. Varnish compounds herein can include both hard and soft varnish compounds. Hard varnish is also referred to as lacquer. Lacquer is typically translucent, light brown in color, and shiny in appearance. Soft varnish is also referred to as sludge. Sludge is typically opaque, soft and dull in appearance. Embodiments herein can detect both lacquer and sludge. Referring now to FIG. 4, a set of charts is shown illustrating a change in density of a hydrocarbon fluid over time as varnish compounds are formed. A hydrocarbon oil was allowed to thermally degrade over time and the density of the same was measured using a fluid property sensor. The chart on the left shows an hourly average plot of density (g/cc) and the chart on the right shows a percentage change plot of density. As can be seen with plot lines 402 and 408, the density changes only gradually at first before encountering a period of time 406 and 410 where the rate of change becomes substantially and measurably different. Time periods 406 and 410 when the rate of change in density is different coincides with the onset of varnish compound formation. As such, changes in density and rates of change of density can be used to detect the onset of varnish compound formation.

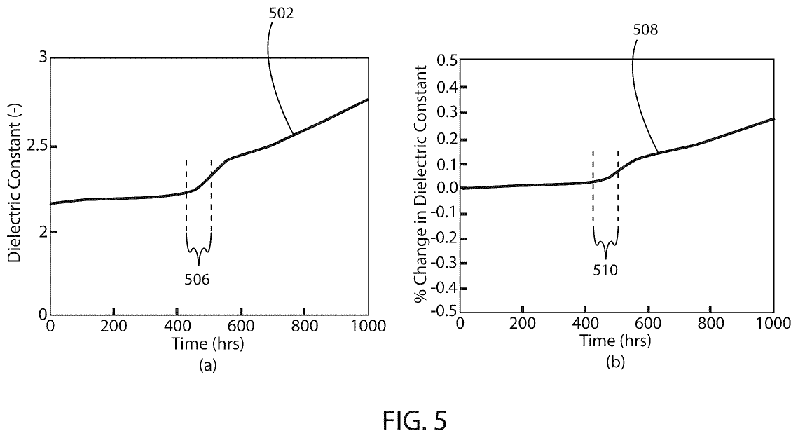

Similarly, FIG. 5 shows a set of charts illustrating a change in dielectric constant of an oil over time as varnish compounds are formed. As before, a hydrocarbon oil was allowed to thermally degrade over time. However, this time, the dielectric constant of the oil was measured using a fluid property sensor. The chart on the left shows an hourly average plot of dielectric constant and the chart on the right shows a percentage change plot of dielectric constant. As can be seen with plot lines 502 and 508, the dielectric constant changes only gradually at first before encountering a period of time 506 and 510 where the rate of change becomes substantially and measurably different. Time periods 506 and 510 when the rate of change in dielectric constant is different coincides with the onset of varnish compound formation. As such, changes in dielectric constant and rates of change of the dielectric constant can be used to detect the onset of varnish compound formation.

As described, systems herein can use the fluid property data and process the same to detect, monitor, and/or detect the onset of formation of varnish compounds. In various embodiments, the system can identify an occurrence of a rate of change in the dielectric constant value indicating an onset of varnish compound formation. In some embodiments, the system can identify a change (in absolute value or rate of change) that crosses a threshold value (predetermined or dynamically determined). In some embodiments, operations performed by the system can include fitting or modeling the measured fluid property value. Fitting or modeling can be performed in various ways. In some embodiments, fitting or modeling can be performed using at least one of hyperbolic, exponential, logistic, sigmoid, or inverse Carreau-Yasuda equations.

Temperature can impact various measured fluid property values, including but not limited to density, dielectric constant, or the like. In various embodiments, the system can be configured to measure temperature of the fluid with a fluid property sensor and/or a discrete temperature sensor. In some embodiments, the system can be configured to normalize measured fluid property values based on the measured fluid temperature.

In some cases, the system can predict the onset of varnish compound formation based on signals from a fluid property sensor. For example, in some cases, a change (in the absolute or in a rate of change) of a fluid property value herein that may not rise to the level of the presence of substantial varnish compounds can be used to predict the imminent onset of substantial varnish compound formation. In some cases, the system can predict the onset of varnish compound formation based on signals from a fluid property sensor along with one or more other pieces of data including one or more of the amount of time that that fluids have been in use, cumulative heat exposure for the fluids, identity or type of the fluids, amounts of anti-oxidant compounds in the fluids, and the like. In some embodiments, pattern matching approaches described below can be used to form predictions of the timing of the onset of varnish compound formation using such pieces of data. In some embodiments, threshold values for fluid properties in the absolute or rates of change in the same can be adjusted based on one or more other pieces of data including one or more of the amount of time that that fluids have been in use, cumulative heat exposure for the fluids, identity or type of the fluids, amounts of anti-oxidant compounds in the fluids, and the like.

In some embodiments, systems herein can process the fluid property data to look for transitions in linear change in the fluid property data to non-linear change to linear again. Such changes from linear to non-linear can be detected by the system using statistical methods, exact methods, machine learning methods, and/or hybrid methods.

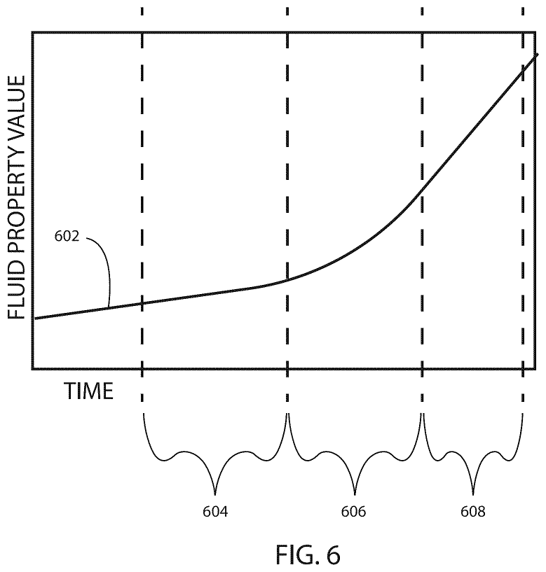

Referring now to FIG. 6, a chart is shown illustrating a fluid property value over time exhibiting linear and non-linear rates of change. In specific, FIG. 6 shows a fluid property value 602 (such as density, dielectric constant, or other fluid properties herein). The fluid property value 602 exhibits a first period with linear rate of change 604, a period with non-linear rate of change 606, and a second period with linear rate of change 608. In various embodiments, the second linear rate of change is greater (e.g., a steeper slope in FIG. 6) than the first linear rate of change. Systems herein can identify each of these rates of change and/or periods of time within the fluid property data.

In some embodiments, the first transition (such as from a linear to a non-linear rate of change) can indicate varnish compounds beginning to deposit, while the second transition (such as from a non-linear to a second linear rate of change) can indicate a critical level of varnish that requires action. The first transition indicating varnish compounds beginning to deposit can be designated as a “watch” level. The second transition indicating a critical level of varnish compounds can be designated as a “warning” level).

Watch levels and warning levels herein can be associated with various system actions. By way of example, in some embodiments, the occurrence of a watch level or a warning level can trigger communications or system alerts for a system user and/or for other connected systems. In some embodiments, the occurrence of a watch level or a warning level can cause the system to provide a recommendation on system maintenance actions including varnish mitigation steps and/or recommended timing for the same.

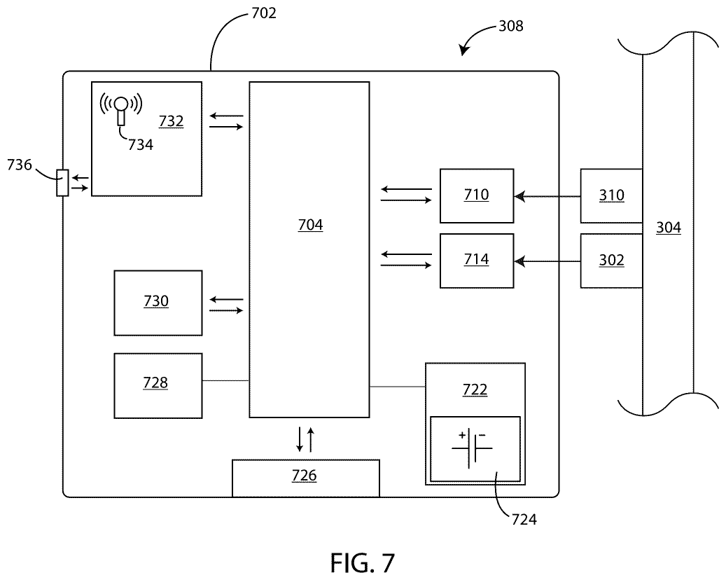

It will be appreciated that systems herein can include various components. Referring now to FIG. 7, a block diagram view of some exemplary components of a varnish detection and/or monitoring system is shown in accordance with various embodiments herein. It will be appreciated, however, that a greater or lesser number of components can be included with various embodiments and that this schematic diagram is merely illustrative. FIG. 7 shows a control unit 308 and a fluid line 304. The control unit 308 can include a housing 702 and a control circuit 704 disposed therein.

The control circuit 704 can include various electronic components including, but not limited to, a microprocessor, a microcontroller, a FPGA (field programmable gate array) chip, an application specific integrated circuit (ASIC), one or more digital signal processing chips, or the like.

In various embodiments, the monitoring system can include a fluid property sensor unit 302 and a fluid property sensor unit channel interface 714. In various embodiments, the monitoring system can also include a secondary sensor unit 310 and a secondary sensor channel interface 714. In some embodiments, the secondary sensor unit 310 can, specifically, be or include a temperature sensor. The sensors can be configured and mounted to detect fluid conditions within a fluid line 304 and can allow in-line and/or on-line sampling and varnish compound detection. The fluid line 304 can, in some cases, form part of a device such as a pump, a valve, a filter housing, a fluid circuit, a lubrication system, a cooling system, or the like. The fluid line 304 can include, in some embodiments, a hydraulic fluid conduit, a lubricating oil conduit, a brake fluid conduit, a refrigerant fluid conduit, a fuel supply conduit, or another type of fluid flow conduit, amongst others.

The channel interfaces can include various components such as amplifiers, analog-to-digital converters (ADCs), digital-to-analog converters (DACs), digital signal processors (DSPs), filters (high-pass, low-pass, band-pass) and the like. In some cases, the channel interfaces may not exist as discrete components but, rather, can be integrated into the control circuit 704.

Fluid property sensors herein can be of various types. Fluid property sensors can include, but are not limited to, those that can measure one or more of dielectric constant, density, resistivity, and viscosity. Sensors that can measure one or more fluid properties can be based on various operating principles including capacitive sensors, coaxial sensors, TDS (total dissolved solids) sensors, and the like. In some embodiments, a fluid property sensor herein can measure multiple fluid properties. In some embodiments, a fluid property sensor herein can be one that is a tuning fork type fluid property sensor, such as a quartz tuning fork type sensor. Exemplary fluid property sensors are commercially available from Parker US, TE Connectivity, and the like. In some cases, fluid property values as measured by sensors herein (such as tuning fork type sensors) can be impacted by varnish compounds such that the measured values may deviate from true values for such fluid properties. However, such deviations can be reflected in rates of change observed herein and can actually contribute to the unique ability of systems herein to detect varnish compound formation herein.

Temperature sensors herein can be of various types. In some embodiments, the temperature sensor can be a thermistor, a resistance temperature device (RTD), a thermocouple, a semiconductor temperature sensor, or the like.

Other secondary sensors herein can include, but are not limited to, pressure sensors, optical sensors, pH sensors, ultrasonic sensors, and the like.

The processing power of the control circuit 704 and components thereof can be sufficient to perform various operations including various operations on signals/data from sensors (such as sensors 302 and 310) including, but not limited to averaging, time-averaging, statistical analysis, normalizing, aggregating, sorting, deleting, traversing, transforming, condensing (such as eliminating selected data and/or converting the data to a less granular form), compressing (such as using a compression algorithm), merging, inserting, time-stamping, filtering, discarding outliers, calculating trends and trendlines (linear, logarithmic, polynomial, power, exponential, moving average, etc.), normalizing data/signals, and the like. Fourier analysis can decompose a physical signal into a number of discrete frequencies, or a spectrum of frequencies over a continuous range. In various embodiments herein, operations on signals/data can include Fast Fourier Transformations (FFT) to convert data/signals from a time domain to a frequency domain. Other operations on signals/data here can include spectral estimation, frequency domain analysis, calculation of root mean square acceleration value (GRMS), calculation of acceleration spectral density, power spectral densities, Fourier series, Z transforms, resonant frequency determination, harmonic frequency determination, and the like. It will be appreciated that while various of the operations described herein (such as Fast Fourier transforms) can be performed by general-purpose microprocessors, they can also be performed more efficiently by digital signal processors (DSPs) which can, in some embodiments, be integrated with the control circuit 704 or may exist as separate, discrete components.

Normalizing operations performed by the control circuit 704 can include, but are not limited to, adjusting one or more values based on another value or set of values. In some embodiments herein, normalizing operations can specifically include normalizing the fluid property values based on temperature or other pieces of data.

In various embodiments, the monitoring system can include a power supply circuit 722. In some embodiments, the power supply circuit 722 can include various components including, but not limited to, a battery 724, a capacitor, a power-receiver such as a wireless power receiver, a transformer, a rectifier, and the like.

In various embodiments the monitoring system can include an output device 726. The output device 726 can include various components for visual and/or audio output including, but not limited to, lights (such as LED lights), a display screen, a speaker, and the like. In some embodiments, the output device can be used to provide notifications or alerts to a system user such as current system status, an indication of a problem, a required user intervention, a proper time to perform a maintenance action, or the like.

In various embodiments the monitoring system can include memory 728 and/or a memory controller. The memory can include various types of memory components including dynamic RAM (D-RAM), read only memory (ROM), static RAM (S-RAM), disk storage, flash memory, EEPROM, battery-backed RAM such as S-RAM or D-RAM and any other type of digital data storage component. In some embodiments, the electronic circuit or electronic component includes volatile memory. In some embodiments, the electronic circuit or electronic component includes non-volatile memory. In some embodiments, the electronic circuit or electronic component can include transistors interconnected to provide positive feedback operating as latches or flip flops, providing for circuits that have two or more metastable states, and remain in one of these states until changed by an external input. Data storage can be based on such flip-flop containing circuits. Data storage can also be based on the storage of charge in a capacitor or on other principles. In some embodiments, the non-volatile memory 728 can be integrated with the control circuit 704.

In various embodiments the monitoring system can include a clock circuit 730. In some embodiments, the clock circuit 730 can be integrated with the control circuit 704. While not shown in FIG. 7, it will be appreciated that various embodiments herein can include a data/communication bus to provide for the transportation of data between components such as an I₂C, a serial peripheral interface (SPI), a universal asynchronous receiver/transmitter (UART), or the like. In some embodiments, an analog signal interface can be included. In some embodiments, a digital signal interface can be included.

In various embodiment the monitoring system can include a communications circuit 732. In various embodiments, the communications circuit can include components such as an antenna 734, amplifiers, filters, digital to analog and/or analog to digital converters, and the like. In some embodiments, the monitoring system can also include wired input/out interface 736 for wired communication with other systems/components including, but not limited to a system or vehicle ECU, a CANBUS network (controller area network), another type of data network, or the like.

### Methods

Many different methods are contemplated herein, including, but not limited to, methods of making systems herein, methods of using systems herein, methods of detecting and/or monitoring varnish compounds, methods of predicting the onset of varnish compound formation, methods of preventing system damage associated with varnish compounds, and the like. Aspects of system/device operation described at various points herein can be performed as operations of one or more methods in accordance with various embodiments herein.

Further, in various embodiments, operations described herein and method steps can be performed as part of a computer-implemented method executed by one or more processors of one or more computing devices. In various embodiments, operations described herein and method steps can be implemented instructions stored on a non-transitory, computer-readable medium that, when executed by one or more processors, cause a system to execute the operations and/or steps.

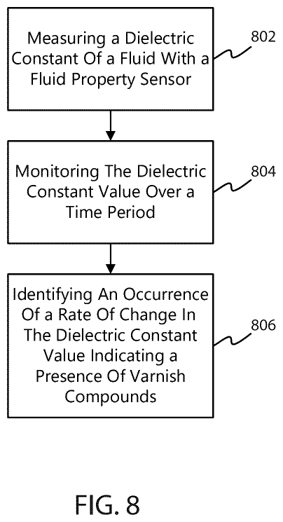

In an embodiment, a method of monitoring varnish compounds in fluids is included, the method measuring a dielectric constant of a fluid with a fluid property sensor, monitoring the dielectric constant value over a time period, and identifying an occurrence of a rate of change in the dielectric constant value indicating a presence of the varnish compounds. Referring now to FIG. 8, a flowchart of operations is shown in accordance with various embodiments herein. In specific, FIG. 8 shows a process including operations of measuring a dielectric constant of a fluid with a fluid property sensor 802, monitoring the dielectric constant value over a time period 804, and identifying an occurrence of a rate of change in the dielectric constant value indicating a presence of varnish compounds 806.

In various embodiments, methods herein can further include identifying an occurrence of the rate of change in the dielectric constant value indicating an onset of varnish compound formation.

In various embodiments, methods herein can further include fitting or modeling the measured dielectric constant value. In an embodiment of the method, fitting or modeling the measured dielectric constant value is performed using at least one of a hyperbolic, exponential, logistic, sigmoid, or an inverse Carreau-Yasuda equation.

In various embodiments, methods herein can further include measuring temperature of the fluid with a temperature sensor. In various embodiments, methods herein can further include normalizing the measured dielectric constant value based on the measured fluid temperature.

In various embodiments, methods herein can further include placing the fluid property sensor after a high heat transfer location in a fluid circuit or a location with a high differential temperature. In an embodiment of the method, the high heat transfer location in a fluid circuit is a heat exchanger in a fluid circuit. In an embodiment of the method, the high heat transfer location in a fluid circuit is a cooler in a fluid circuit, such as a fan type cooler.

In an embodiment of the method, the fluid is a hydrocarbon fluid. In an embodiment of the method, the fluid is a lubrication fluid. In an embodiment of the method, the fluid is a hydraulic fluid. In an embodiment of the method, the fluid is a gas turbine oil.

In various embodiments, methods herein can further include issuing an alert or communication when varnish compounds are detected. In various embodiments, methods herein can further include initiating varnish compound removal and/or mitigation when varnish compounds are detected. Operations to remove or mitigate varnish compounds can include one or more of the replacement of fluids with new fluids lacking varnish compounds, use of an adsorbent filter to remove the varnish compounds, shunting fluid flow through an adsorbent filter to remove the varnish compounds, using an electrostatic separation device to remove varnish compounds, adding antioxidant compounds (aromatic amines, phenolics-or hindered phenols, phosphites, thioethers, thioesters, and the like) to slow varnish compound formation, incorporating varnish-removing additives into the working fluid, and the like.

In various embodiments, methods herein can further include detecting varnish compound removal and/or remediation after a removal or mitigation operation.

In various embodiments, methods herein can further include measuring a density of the fluid as an additional property with the fluid property sensor, monitoring the density over a time period, and identifying an occurrence of a rate of change in the density value indicating a presence of varnish compounds.

In various embodiments, methods herein can further include measuring a resistivity of the fluid as an additional property with the fluid property sensor, monitoring the resistivity over a time period, and identifying an occurrence of a rate of change in the resistivity value indicating a presence of varnish compounds.

In various embodiments, methods herein can further include measuring a viscosity of the fluid as an additional property with the fluid property sensor, monitoring the viscosity over a time period, and identifying an occurrence of a rate of change in the viscosity value indicating a presence of varnish compounds.

In various embodiments, methods herein can further include measuring a non-fluid property, monitoring the non-fluid property over a time period, and identifying an occurrence of a change in the non-fluid property value indicating a presence of varnish compounds.

In an embodiment, the non-fluid property can include a differential pressure value of a system filter.

In an embodiment of the method, the varnish compounds comprise hard varnish compounds (or lacquer compounds). In an embodiment of the method, the varnish compounds comprise soft varnish compounds (or sludge).

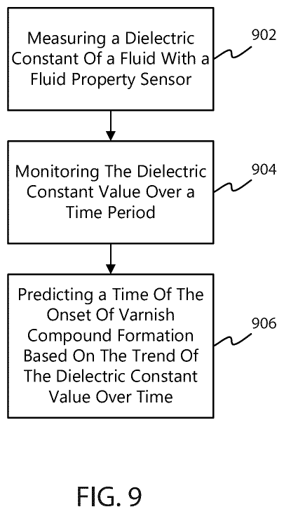

In an embodiment, a method of monitoring varnish compounds in fluids is included, the method measuring a dielectric constant of a fluid with a fluid property sensor, monitoring the dielectric constant value over a time period, and predicting a time of the onset of varnish compound formation based on the trend of the dielectric constant value over time. Referring now to FIG. 9, a flowchart of operations is shown in accordance with various embodiments herein. In specific, FIG. 9 shows a process including operations of measuring a dielectric constant of a fluid with a fluid property sensor 902, monitoring the dielectric constant value over a time period 904, and predicting a time of the onset of varnish compound formation based on the trend of the dielectric constant value over time 906.

In various embodiments, methods herein can further include estimating an amount of time it will take for the dielectric constant to hit a particular value based on the trend of the dielectric constant value over time.

In an embodiment of the method, the particular value is predetermined or dynamically determined.

In various embodiments, methods herein can further include estimating an amount of time it will take for the dielectric constant to hit a particular value based on a comparison of the trend of the dielectric constant value over time against previously stored data regarding how dielectric constant values change over time for a comparable fluid and/or a comparable system in which the fluid resides.

In various embodiments, methods herein can further include matching the trend of the dielectric constant value over time against a set of previously stored patterns reflecting different time periods until an onset of varnish compound formation begins.

In various embodiments, methods herein can further include identifying an occurrence of a rate of change in the dielectric constant value indicating an onset of varnish compound formation.

In various embodiments, methods herein can further include issuing an alert or communication based on the predicted time of the onset of varnish compound formation.

In various embodiments, methods herein can further include initiating varnish compound removal and/or mitigation at a particular time based on the predicted time of the onset of varnish compound formation.

In various embodiments, methods herein can further include detecting varnish compound removal and/or remediation after a removal or mitigation operation.

In various embodiments, methods herein can further include measuring a density of the fluid as an additional property with the fluid property sensor, monitoring the density over a time period, and predicting a time of the onset of varnish compound formation based on the trend of the density value over time.

In various embodiments, methods herein can further include measuring a resistivity of the fluid as an additional property with the fluid property sensor, monitoring the resistivity over a time period, and predicting a time of the onset of varnish compound formation based on the trend of the resistivity value over time.

In various embodiments, methods herein can further include measuring a viscosity of the fluid as an additional property with the fluid property sensor, monitoring the viscosity over a time period, and predicting a time of the onset of varnish compound formation based on the trend of the viscosity value over time.

In various embodiments, methods herein can further include measuring a non-fluid property, monitoring the non-fluid property over a time period, and predicting a time of the onset of varnish compound formation based on the trend of the non-fluid property value over time.

In an embodiment, a method of monitoring varnish compounds in fluids is included, the method measuring a density of a fluid with a fluid property sensor, monitoring the density over a time period, and identifying an occurrence of a rate of change in the density value indicating a presence of varnish compounds.

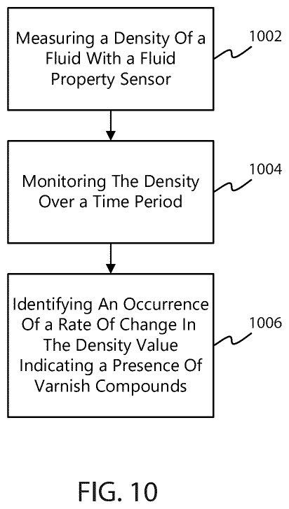

Referring now to FIG. 10, a flowchart of operations is shown in accordance with various embodiments herein. In specific, FIG. 10 shows a process including operations of measuring a density of a fluid with a fluid property sensor 1002, monitoring the density over a time period 1004, and identifying an occurrence of a rate of change in the density value indicating a presence of varnish compounds 1006.

In various embodiments, methods herein can further include identifying an occurrence of the rate of change in the density value indicating an onset of varnish compound formation.

In various embodiments, methods herein can further include measuring temperature of the fluid with a temperature sensor. In various embodiments, methods herein can further include normalizing the measured density value based on the measured fluid temperature.

In various embodiments, methods herein can further include identifying when a measured fluid density value exceeds that of water.

In various embodiments, methods herein can further include measuring a dielectric constant of the fluid with the fluid property sensor, monitoring the dielectric constant over a time period, and identifying an occurrence of a rate of change in the dielectric constant value indicating a presence of varnish compounds.

In various embodiments, methods herein can further include measuring a resistivity of the fluid with the fluid property sensor, monitoring the resistivity over a time period, and identifying an occurrence of a rate of change in the resistivity value indicating a presence of varnish compounds.

In various embodiments, methods herein can further include measuring a viscosity of the fluid with the fluid property sensor, monitoring the viscosity over a time period, and identifying an occurrence of a rate of change in the viscosity value indicating a presence of varnish compounds.

In an embodiment, a method of monitoring varnish compounds in fluids is included, the method measuring a density of a fluid with a fluid property sensor, monitoring the density value over a time period, and predicting a time of the onset of varnish compound formation based on the trend of the density value over time.

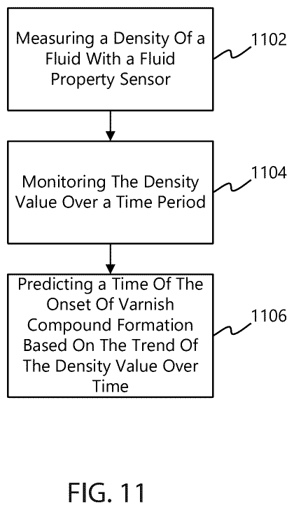

Referring now to FIG. 11, a flowchart of operations is shown in accordance with various embodiments herein. In specific, FIG. 11 shows a process including operations of measuring a density of a fluid with a fluid property sensor 1102, monitoring the density value over a time period 1104, and predicting a time of the onset of varnish compound formation based on the trend of the density value over time 1106.

In various embodiments, methods herein can further include estimating an amount of time it will take for the density to hit a particular value based on the trend of the density value over time. In an embodiment of the method, the particular value is predetermined or dynamically determined.

In various embodiments, methods herein can further include estimating an amount of time it will take for the density to hit a particular value based on a comparison of the trend of the density over time against previously stored data regarding how density values change over time for a comparable fluid and/or a comparable system in which the fluid resides.

In various embodiments, methods herein can further include matching the trend of the density value over time against a set of previously stored patterns reflecting different time periods until an onset of varnish compound formation begins.

In various embodiments, methods herein can further include identifying an occurrence of a rate of change in the density value indicating an onset of varnish compound formation.

In various embodiments, methods herein can further include measuring a resistivity of the fluid with the fluid property sensor, monitoring the resistivity over a time period, and predicting a time of the onset of varnish compound formation based on the trend of the resistivity value over time.

In various embodiments, methods herein can further include measuring a dielectric constant of the fluid with the fluid property sensor, monitoring the dielectric constant over a time period, and predicting a time of the onset of varnish compound formation based on the trend of the dielectric constant value over time.

In various embodiments, methods herein can further include measuring a viscosity of the fluid with the fluid property sensor, monitoring the viscosity over a time period, and predicting a time of the onset of varnish compound formation based on the trend of the viscosity value over time.

In an embodiment, a method of monitoring varnish compounds in fluids is included, the method measuring a resistivity of a fluid with a fluid property sensor, monitoring the resistivity over a time period, and identifying an occurrence of a rate of change in the resistivity value indicating a presence of varnish compounds.

In various embodiments, methods herein can further include identifying an occurrence of the rate of change in the resistivity value indicating an onset of varnish compound formation.

In various embodiments, methods herein can further include measuring temperature of the fluid with a temperature sensor. In various embodiments, methods herein can further include normalizing the measured resistivity value based on the measured fluid temperature.

In various embodiments, methods herein can further include measuring a dielectric constant of the fluid with the fluid property sensor, monitoring the dielectric constant over a time period, and identifying an occurrence of a rate of change in the dielectric constant value indicating a presence of varnish compounds.

In various embodiments, methods herein can further include measuring a density of the fluid with the fluid property sensor, monitoring the density over a time period, and identifying an occurrence of a rate of change in the density value indicating a presence of varnish compounds.

In various embodiments, methods herein can further include measuring a viscosity of the fluid with the fluid property sensor, monitoring the viscosity over a time period, and identifying an occurrence of a rate of change in the viscosity value indicating a presence of varnish compounds.

In an embodiment, a method of monitoring varnish compounds in fluids is included, the method including measuring a resistivity of a fluid with a fluid property sensor, monitoring the resistivity value over a time period, and predicting a time of the onset of varnish compound formation based on the trend of the resistivity value over time.

In various embodiments, methods herein can further include estimating an amount of time it will take for the density to hit a particular value based on the trend of the resistivity value over time. In an embodiment of the method, the particular value is predetermined or dynamically determined.

In various embodiments, methods herein can further include estimating an amount of time it will take for the resistivity to hit a particular value based on a comparison of the trend of the resistivity over time against previously stored data regarding how density values change over time for a comparable fluid and/or a comparable system in which the fluid resides.

In various embodiments, methods herein can further include matching the trend of the resistivity value over time against a set of previously stored patterns reflecting different time periods until an onset of varnish compound formation begins.

In various embodiments, methods herein can further include identifying an occurrence of a rate of change in the resistivity value indicating an onset of varnish compound formation.

In various embodiments, methods herein can further include measuring temperature of the fluid with a temperature sensor. In various embodiments, methods herein can further include normalizing the measured resistivity value based on the measured fluid temperature.

In various embodiments, methods herein can further include measuring a dielectric constant of the fluid with the fluid property sensor, monitoring the dielectric constant over a time period, and predicting a time of the onset of varnish compound formation based on the trend of the dielectric constant value over time.

In various embodiments, methods herein can further include measuring a density of the fluid with the fluid property sensor, monitoring the density over a time period, and predicting a time of the onset of varnish compound formation based on the trend of the density value over time.

In various embodiments, methods herein can further include measuring a viscosity of the fluid with the fluid property sensor, monitoring the viscosity over a time period, and predicting a time of the onset of varnish compound formation based on the trend of the viscosity value over time in combination with other data herein.

In various embodiments, methods herein can further include measuring a non-fluid property, monitoring the non-fluid property over a time period, and predicting a time of the onset of varnish compound formation based on the trend of the non-fluid property value over time.

### Pattern/Template Generation and Pattern Matching

It will be appreciated that in various embodiments herein, the system can be used to detect a pattern or patterns in signals indicative of the state of varnish presence and/or formation. Such patterns can be detected in various ways. Some techniques are described elsewhere herein, but some further examples will now be described.

In various embodiments, the system can be configured to detect varnish compounds and/or an onset of formation of the same. In some embodiments, varnish compounds can be identified based on identifying or matching characteristic patterns in the data from a fluid property sensor, and/or other sensors. For example, a “positive” pattern for sensor data associated with a varnish presence or formation onset event or state can be stored by the system and current sensor data can be periodically matched against such a pattern. If a match exceeding a threshold value is found, then an operational event or state can be deemed to have taken place. As another example, a “negative” pattern for sensor data associated with a particular operational event or state can be stored by the system and current data can be periodically matched against such a pattern.

In some embodiments, one or more sensors (such as a fluid property sensor herein or the like) can be operatively connected to a controller (such as the control circuit 704 described in FIG. 7) or another processing resource (such as a processor of another device or a processing resource in the cloud). The control circuit 704 or other processing resource can be adapted to receive data representative of a fluid state parameter from one or more of the sensors and/or determine statistics of the system over a monitoring time period based upon the data received from the sensor(s). As used herein, the term “data” can include a single datum or a plurality of data values or statistics. The term “statistics” can include any appropriate mathematical calculation or metric relative to data interpretation, e.g., probability, confidence interval, distribution, range, or the like. Further, as used herein, the term “monitoring time period” means a period of time over which signal data is measured and statistics are determined. The monitoring time period can be any suitable length of time, e.g., 1 second, 10 seconds, 30 seconds, 1 minute, 10 minutes, 30 minutes, 1 hour, 1 day, 1 week, 1 month, etc., or a range of time between any of the foregoing time periods.

Any suitable technique or techniques can be utilized to determine statistics for the various data from the sensors, e.g., direct statistical analyses of time series data from the sensors, differential statistics, comparisons to baseline or statistical models of similar data, etc. Such techniques can be general or system-specific and represent long-term or short-term operational behavior. These techniques could include standard pattern classification methods such as Gaussian mixture models, clustering as well as Bayesian approaches, machine learning approaches such as neural network models and deep learning, and the like, and/or combinations of at least two techniques.

Further, in some embodiments, the controller or control circuit 704 can be adapted to compare data, data features, and/or statistics against various other patterns, which could be predetermined or starting patterns (baseline patterns) based on the type or model of the filtration system, one or more predetermined patterns that serve as patterns indicative of an occurrence of an event or state of the presence or onset of formation of varnish compounds (positive example patterns), one or more predetermined patterns that service as patterns indicative of the absence of an operational event or state (negative example patterns), or the like. As merely one scenario, if a pattern is detected for one or more fluid property parameters that exhibits similarity crossing a threshold value to a particular positive example pattern or substantial similarity to that pattern, wherein the pattern is specific for an event or state of the presence of varnish and/or the onset of formation of the same, then that can be taken as an indication that an occurrence of the event or state has occurred.

Similarity and dissimilarity can be measured directly via standard statistical metrics such normalized Z-score, or similar multidimensional distance measures (e.g., Mahalanobis or Bhattacharyya distance metrics), or through similarities of modeled data and machine learning. These techniques can include standard pattern classification methods such as Gaussian mixture models, clustering as well as Bayesian approaches, neural network models, deep learning, and/or a combination of at least two techniques.

As used herein the term “substantially similar” means that, upon comparison, the sensor data are congruent or have statistics fitting the same statistical model, each with an acceptable degree of confidence. The threshold for the acceptability of a confidence statistic may vary depending upon the filtration system, sensor(s), sensor arrangement, type of data, context, condition, etc.

The statistics associated with the status of varnish presence and/or the onset of formation of varnish over the monitoring time period, can be determined by utilizing any suitable technique or techniques, e.g., standard pattern classification methods such as Gaussian mixture models, clustering, hidden Markov models, as well as Bayesian approaches, neural network models, and deep learning, and/or a combination of at least two techniques.

Various embodiments herein specifically include the application of a machine learning classification model. In various embodiments, the system device can be configured to periodically update the machine learning classification model based on indicators of particular varnish presence and/or formation events. In some embodiments, user input can be used to positively identify particular events and then this information can be used as part of a supervised machine learning approach to positively characterize patterns associated with particular varnish presence and/or formation events or states. For example, if it is known that varnish is present and/or the onset of varnish formation has occurred, a user can input this information into the system and then data corresponding in time with the formation of varnish compounds can be processed in order to generate a pattern that is indicative of the formation of varnish compounds.

In some embodiments, a training set of data can be used to generate a machine learning classification model. The input data can include fluid state data described herein as tagged/labeled with binary and/or non-binary classifications of particular states of varnish presence, amounts of varnish, the onset of varnish formation, and the like. Binary classification approaches can utilize techniques including, but not limited to, logistic regression, k-nearest neighbors, decision trees, support vector machine approaches, naive Bayes techniques, and the like. Multi-class classification approaches (e.g., for non-binary classifications of stress) can include k-nearest neighbors, decision trees, naive Bayes approaches, random forest approaches, and gradient boosting approaches amongst others.

It should be noted that, as used in this specification and the appended claims, the singular forms “a,” “an,” and “the” include plural referents unless the content clearly dictates otherwise. It should also be noted that the term “or” is generally employed in its sense including “and/or”unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A method of monitoring varnish compounds in fluids comprising:
measuring a dielectric constant of a fluid with a fluid property sensor;
monitoring the dielectric constant value over a time period; and
identifying an occurrence of a rate of change in the dielectric constant value indicating a presence of the varnish compounds.

2. The method of monitoring varnish compounds in fluids of claim 1, further comprising identifying an occurrence of the rate of change in the dielectric constant value indicating an onset of varnish compound formation.

3-4. (canceled)

5. The method of monitoring varnish compounds in fluids of claim 1, further comprising identifying a period of a linear rate of change in the fluid property followed by a period of a non-linear rate of change in the fluid property.

6. The method of monitoring varnish compounds in fluids of claim 5, further comprising identifying a second period of a linear rate of change in the fluid property following the period of non-linear rate of change.

7. The method of monitoring varnish compounds in fluids of claim 5, wherein the fluid property is dielectric constant.

8-9. (canceled)

10. The method of monitoring varnish compounds in fluids of claim 1, further comprising placing the fluid property sensor after a high heat transfer location in a fluid circuit or at a location with a high differential temperature in a fluid circuit.

11. The method of monitoring varnish compounds in fluids of claim 10, wherein the high heat transfer location in a fluid circuit is a heat exchanger or cooler in a fluid circuit.

12. The method of monitoring varnish compounds in fluids of claim 1, wherein the fluid property sensor is a tuning fork-type fluid property sensor.

13-16. (canceled)

17. The method of monitoring varnish compounds in fluids of claim 1, further comprising issuing an alert or communication when varnish compounds are detected.

18. The method of monitoring varnish compounds in fluids of claim 1, further comprising initiating varnish compound removal and/or mitigation when varnish compounds are detected.

19. The method of monitoring varnish compounds in fluids of claim 1, further comprising detecting varnish compound removal and/or remediation after a removal or mitigation operation.

20. The method of monitoring varnish compounds in fluids of claim 1, further comprising:
measuring a density of the fluid as an additional property with the fluid property sensor;
monitoring the density over a time period; and
identifying an occurrence of a rate of change in the density value indicating a presence of varnish compounds.

21. The method of monitoring varnish compounds in fluids of claim 1, further comprising:
measuring a resistivity of the fluid as an additional property with the fluid property sensor,
monitoring the resistivity over a time period; and
identifying an occurrence of a rate of change in the resistivity value indicating a presence of varnish compounds.

22. The method of monitoring varnish compounds in fluids of claim 1, further comprising:
measuring a viscosity of the fluid as an additional property with the fluid property sensor;
monitoring the viscosity over a time period; and
identifying an occurrence of a rate of change in the viscosity value indicating a presence of varnish compounds.

23. The method of monitoring varnish compounds in fluids of claim 1, further comprising:
measuring a non-fluid property;
monitoring the non-fluid property over a time period; and
identifying an occurrence of a change in the non-fluid property value indicating a presence of varnish compounds.

24. The method of monitoring varnish compounds in fluids of claim 23, the non-fluid property comprising a differential pressure value of a system filter.

25-26. (canceled)

27. A system for monitoring varnish compounds in fluids comprising:
a control circuit; and
a fluid property sensor;
wherein the system for monitoring varnish compounds in fluids is configured to
measure a dielectric constant of a fluid with the fluid property sensor;
monitor the dielectric constant over a time period; and
identify an occurrence of a rate of change in the dielectric constant value indicating a presence of the varnish compounds.

28-51. (canceled)

52. A method of monitoring varnish compounds in fluids comprising:
measuring a dielectric constant of a fluid with a fluid property sensor;
monitoring the dielectric constant value over a time period; and
predicting a time of the onset of varnish compound formation based on the trend of the dielectric constant value over time.

53-205. (canceled)
